(19) United States
(12) Patent Application Publication
(10) Pub. No.: US 2014/0179554 A1
(43) Pub. Date: Jun. 26, 2014

(54) Microarrays

(71) Applicant: Hutman Diagnostics AG, Basel (CH)

(72) Inventors: Michal Svoboda, Muttenz (CH); Xenia Svoboda, Muttenz (CH)

(73) Assignee: Hutman Diagnostics AG, Basel (CH)

(21) Appl. No.: 14/136,313

(22) Filed: Dec. 20, 2013

Related U.S. Application Data
(60) Provisional application No. 61/745,020, filed on Dec. 21, 2012.

(51) Int. Cl. C12Q 1/68

(52) U.S. Cl. CPC C12Q 1/6888
USPC 506/9; 506/16

(57) ABSTRACT

Provided herein are microarrays (protein and/or nucleic acid microarrays) containing an array of spots on a solid substrate, wherein the spots are arranged to reduce the risk of array misalignment and/or to facilitate the visual interpretation of an array image by a human operator. Also provided herein are methods of using such arrays and kits containing such arrays.

### RELATED APPLICATIONS

This application claims the benefit of priority to U.S. Provisional Patent Application Ser. No. 61/745,020 filed Dec. 21, 2012, which is hereby incorporated by reference in its entirety.

### BACKGROUND

Microarrays are a useful tool for analysing the gene expression, genetic mutations, and detecting pathogens. Such microarrays are commonly prepared as square arrays of spots, with each spot containing nucleic acid probes or antibodies that are able to bind to a specific target. Binding of the target to a spot on the array is detected using a detectable label that is attached to the target either before or after contacting the target with the microarray.

For example, nucleic acid microarrays are generally formed as a square array of spots, each containing a nucleic acid probe having a complementary sequence to a target of interest. Such arrays are contacted with a solution containing detectably labelled target nucleic acids. The target nucleic acid will become immobilized on a particular spot if it contains a probe with a complementary nucleic acid sequence. After washing away non-immobilized nucleic acids, a suitable reader can be used to detect the presence of the immobilized nucleic acids using the detectable label. For example, a fluorescence reader can be used to detect spots on which fluorescently labelled targets are immobilized. Alternatively, targets labelled with certain enzymes can be contacted with a chromogenic substrate and the resulting coloration change can be read as an absorbance value by a suitable device.

Low density DNA, protein or mixed DNA/protein microarrays are useful for the simultaneous detection of multiple pathogens. The spots on such an array are usually between 50 and 150 micrometres in diameter, and therefore clearly visible with minimal magnification. In a typical application, the fluorescence, (chemi)luminescence or absorbance signals of the positive array spots are read by a suitable reader, and the resulting data is interpreted with a computer program. When a specific pathogen is present in the sample, usually only a few spots are fluorescing, and no spots may fluoresce with a negative sample. Minor defects in the array, like dried droplets of liquid, dust particles or haze, which would not prevent a human from determining whether a spot is positive or negative can render the array unintelligible for a machine. This aspect of computer-aided pathogen detection creates a requirement for high quality arrays, further demanding a great deal of care to be taken with array handling and reading.

An additional risk associated with the use of microarrays is that the image of the microarray is inadvertently rotated and/or flipped, thereby producing erroneous results. With high-density genomic arrays, the array holder and dedicated instrumentation are often specially designed such that the array only fits into the instrument in a single orientation in order to safeguard against the array misreading. However, no such safeguard exists for low-density arrays read with generic laboratory equipment.

Thus, there exists a great need for improved microarrays that reduce the risk of array misalignment and/or facilitate the visual interpretation of an array image by a human operator.

### SUMMARY

Provided herein are microarrays (protein and/or nucleic acid microarrays) containing an array of spots on a solid substrate, wherein the spots are arranged to reduce the risk of array misalignment and/or to facilitate the visual interpretation of an array image by a human operator. Also provided herein are methods of using such arrays and kits containing such arrays.

In certain embodiments, described herein are nucleic acid and/or protein microarrays containing an array of spots on a solid substrate (e.g., a rectangular grid of spots such as a square grid of spots). In some embodiments the array of spots includes a plurality of pathogen-specific spots. Such pathogen-specific spot can contain a pathogen-specific nucleic acid probe or antibody immobilized on the solid substrate. In some embodiments the array of spots includes one or more always-detectable spots containing a detectable substance immobilized to the solid substrate (e.g., an always-fluorescing spot containing a fluorescent dye immobilized on the solid substrate). In some embodiments the array of spots includes one or more never-detectable spots. Such spots, for example, may be empty positions in the array or they may be spotted with spotting buffer that does not contain a detectable substance, a nucleic acid probe or an antibody (e.g., never-fluorescing spots containing neither a fluorescent dye nor a nucleic acid probe immobilized to the solid substrate). In some embodiments the array of spots also includes one or more positive-control spots containing, for example, a nucleic acid probe having a sequence complementary to a positive control nucleic acid (e.g., a conserved eubacterial 16S rRNA sequence).

In certain embodiments, the one or more always-detectable spots and the one or more never-detectable spots are positioned such that the array of spots has neither rotational symmetry nor mirror symmetry. For example, in some embodiments the position of one or more always-detectable spots and one or more never-detectable spots are such that rotation of the microarray by 90 degrees, 180 degrees or 270 degrees results in at least one always-detectable spot being in a position occupied by a never-detectable spot in an un-rotated array. In another embodiment, the position of one or more always-detectable spots and one or more never-detectable spots are such that flipping the microarray on its horizontal or vertical axis results in at least one always-detectable spot being in a position occupied by a never-detectable spot in an un-rotated array. In some embodiments the position of one or more always-detectable spots and one or more never-detectable spots are such that rotation of the microarray by 90 degrees, 180 degrees or 270 degrees and flipping the microarray on its horizontal or vertical axis results in at least one always-detectable spot being in a position occupied by a never-detectable spot in an un-rotated array. In certain embodiments the position of one or more always-detectable spots and one or more never-detectable spots are such that rotation of the microarray by 90 degrees, 180 degrees or 270 degrees results in at least one never-detectable spot being in a position occupied by an always-detectable spot in an un-rotated array. In some embodiments the position of one or more always-detectable spots and one or more never-detectable spots are such that flipping the microarray on its horizontal or vertical axis results in at least one never-detectable spot being in a position occupied by an always-detectable spot in an un-rotated array. In certain embodiments the position of one or more always-detectable spots and one or more never-detectable spots are such that rotation of the microarray by 90 degrees, 180 degrees or 270 degrees and flipping the microarray on its horizontal or vertical axis results in at least one never-detectable spot being in a position occupied by an always-detectable spot in an un-rotated array.

In some embodiments, the microarray is a rectangular grid of spots that is made up of multiple of sub-arrays of spots. In some embodiments the distance between adjacent sub-arrays is different than the distance between adjacent spots within the sub-arrays. For example, in some embodiments the distance between adjacent sub-arrays is greater than the distance between adjacent spots within the sub-arrays. In some embodiments the distance between adjacent sub-arrays is about 1.1 times, 1.2 times, 1.3 times, 1.4 times, 1.5 times, 1.6 times, 1.7 times, 1.8 times, 1.9 times, 2.0 times, 2.5 times, 3 times, 4 times or 5 times the distance between spots within the sub-arrays. In some embodiments, the rectangular grid of spots contains at least 2, 3, 4, 5, 6, 7, 8 or 9 sub-arrays. In certain embodiments the rectangular grid of spots contains 2, 3, 4, 5, 6, 7, 8, 9, 10, 11, 12, 13, 14, 15, 16, 17, 18, 19, 20, 21, 22, 23, 24 or 25 sub-arrays. In some embodiments, the rectangular grid of spots contains 4, 9, 16 or 25 sub-arrays. In some embodiments each sub-array is a square grid of spots.

In some embodiments, the microarray contains a rectangular grid of spots (e.g., a square grid of spots), and an always-fluorescing spot is positioned in at least one corner of the rectangular grid of spots. In some embodiments, always-fluorescing spots are positioned at 2 or 3 corners of the rectangular grid of spots and a never-fluorescing spot is positioned in the other corners of the rectangular grid of spots.

In some embodiments, the microarray contains a plurality of pathogen-specific spots that are organized as one or more identification groups. For example, in some embodiments, in such an identification group, the pathogen-specific nucleic acid probe or antibody contained by each spot within an identification group is specific for a target nucleic acid or protein from a related group of pathogens. In some embodiments, the identification groups contain at least 2, 3, 4, 5, 6, 7 or 8 spots arranged in a square, rectangle and/or line. In some embodiments, the identification groups contain no more than 4, 5, 6, 7, 8, 9, 10, 11, 12, 13, 14, 15, 16, 17, 18, 19 or 20 spots arranged in a square, rectangle and/or line. In some embodiments the related group of pathogens contains no more than 30, 29, 28, 27, 26, 25, 24, 23, 22, 21, 20, 19, 18, 17, 16, 15, 14, 13, 12, 11, 10, 9, 8, 7, 6, 5, 4, 3 or 2 pathogens.

In some embodiments, provided herein is a method of performing a nucleic acid microarray analysis using a microarray described herein. In some embodiments, the method includes the step of contacting a sample with the microarray. In some embodiments, the sample contains nucleic acids and/or proteins. In some embodiments, the nucleic acids or proteins are detectably labeled (e.g., fluorescently labeled). In some embodiments the method includes the step of incubating the microarray under conditions that would permit target proteins and/or target nucleic acids in the sample, if present, to become immobilized on spots of the microarray containing nucleic acid probes or antibodies specific for such target nucleic acids or proteins. In some embodiments the method includes the step of washing the microarray to remove non-immobilized nucleic acids and/or proteins. In some embodiments the method includes the step of detecting the presence of proteins or nucleic acids from the sample immobilized on at least one of the spots of the microarray. In some embodiments, the method includes the step of detecting fluorescence emitted by the spots of the microarray. In some embodiments the method includes the step of contacting the microarray with a chromogenic substrate and detecting a color change of the spots of the microarray. In some embodiments, the method includes performing an amplification reaction (e.g., a PCR reaction) on a nucleic acid of the sample before or after contacting it with the microarray.

In some embodiments, the method includes the step of generating an image of the microarray during the detection step. In certain embodiments the method includes the step of visually interpreting the image of the microarray. In some embodiments the step of visually interpreting the image of the microarray is performed by the operator without the aid of image recognition software.

In some embodiments, provided herein is a kit comprising a microarray described herein. In some embodiments the kit includes a microarray pattern identification aid, such as a rotary dial device and/or a printed pattern identification tree. In some embodiments the kit also includes instructions for using the microarray device.

### BRIEF DESCRIPTION OF THE FIGURES

FIG. 1 shows an array that includes four sub-arrays of three times three spots, the distance between the rows or columns within the sub-arrays (c, a) is different from that between the adjacent sub-arrays (d or b). The array also includes four always-fluorescing spots (always positive, 1a through 1d) and two never fluorescing spots (never positive, 2a and 2b) spots. Spots 3 to 32 are target selective probes.

FIG. 2 shows that the array of FIG. 1 lacks rotational symmetry (a. correct position, b. 90 deg clockwise rotated image, c. 180 deg clockwise rotated image, d. 270 deg clockwise rotated image).

FIG. 3 shows that the array of FIG. 1 lacks mirror-image symmetry (a. correct position, b. mirror image (flipped) of the array, c. 90 deg clockwise rotated flipped image, d. 180 deg clockwise rotated flipped image, d. 270 deg clockwise rotated flipped image).

FIG. 4 shows that if the array of FIG. 1 is misaligned by one row (shifted grid) from the correct position, it results in pivotal elements 1b and 1d not being detected and thus the alignment can be rejected. For the sake of image clarity, the grid is also shifted by about half of a spot diameter.

FIG. 5 shows a layout that includes 4 sectors of 6 times 6 spots with 8 always-detectable spots (1a through 1h), sixteen never-detectable spots (2a through 2p), and eight pathogen-specific spots (3a through 3d, 4a through 4d) that turn positive if the sample contains bacteria targeted by the specific probes or antibodies on the array (other spots, “x”).

FIG. 6 shows dimensions of the array according to Example 1, elements 1(x) are always-fluorescing spots, elements 2(x) are never-fluorescing spots, and elements 3(x) and 4(x) are amplification-control spots (must be on for the result to be valid). All lengths in millimeters. The dimensions indicated are in mm, the respective dimensions are 0.012″ and 0.018″ in US units.

FIG. 7 shows a print layout of the array according to Example 1, individual probes were printed only positions denoted with “x”, other positions were left empty

FIG. 8 shows a scan of the array according to Example 2 hybridised with a positive control sample, location of representative position control elements is emphasised by arrow and circle, 1(x) through 4(x) have the same meaning as in FIGS. 5 and 6.

FIG. 9 shows grid positioning over (9a) properly oriented scan, (9b) scan rotated 90 deg, (9c) scan rotated 180 deg, (9d) scan rotated 270 deg, and (9e) scan flipped horizontally. Representative mismatched positioning elements are emphasised by arrows.

FIG. 10 shows a layout of a micro-array indicating the position of orientation (always-fluorescing spots—1, never-fluorescing spots—2, control spots—3, 4, and specific/multispecific probes (unlabelled positions). The array contains three identical sub-arrays A, B and C to provide robust reading through redundancy.

FIG. 11 shows the probe layout on the Array of Example 3. Only one of the three identical sub-arrays is shown.

FIG. 12 shows the fluorescence patterns for a subset of pathogens. Only one of the three identical sub-arrays is included, white spot indicates intense fluorescence, grey spot very weak to weak fluorescence, no spot indicates no fluorescence. 12A shows an example of the fluorescence patterns for a subset of pathogens selected from the enteric rods group, A—bacteraemia indicating spots (eubacterial universal probes), B—E. coli/Citrobacter spp. group indicator spots, C—E. coli/Citrobacter spp. identification square, D—enteric rods indicating spot (except for Citrobacter and E. coli), E—Klebsiella/Enterobacter identification square. 12B shows an example of the fluorescence patterns for a subset of pathogens—Streptococci, Enterococci and Staphylococcus, A—bacteraemia indicating spots (eubacterial universal probes), B—enterococcus group indicator, C—Enterococcus identification square, D—Streptococcus group indicator, E—Streptococcus identification square, F—Staphylococcus group indicator, G—Staphylococcus identification square. 12C shows an example of the fluorescence patterns for a subset of fungal pathogens—A bacteraemia indicating spots (eubacterial universal probes)—not fluorescing, B—Candida identification square.

FIG. 13 shows the probe layout on the Array of Example 4. Only one of the three identical sub-arrays is shown.

FIG. 14 shows the fluorescence patterns for a subset of pathogens, selected from the pathogen list in Table 5. Only one of the three identical sub-arrays is included, white spot indicates intense fluorescence, grey spot very weak to weak fluorescence, no spot indicates no fluorescence. 14A shows an example of the fluorescence patterns from a subset of pathogens selected from the enteric rods group, A—bacteraemia indicating spots (eubacterial universal probes), B—E. coli specific probes, C—E. coli/Citrobacter spp. group indicator spots, D—Klebsiella/Enterobacter identification field, E—enteric rod multispecific probe (except for Citrobacter and E. coli). 14B shows the fluorescence patterns for a subset of pathogens—Streptococci, Enterococci and Staphylococcus, A—bacteraemia indicating spots (eubacterial universal probes), B—Enterococcus identification area, C—enterococcus group probe, D—Streptococcus group probe, E—Streptococcus identification area, F—Staphylococcus group probes, G—Staphylococcus identification area. 14C shows the fluorescence patterns for a subset of fungal pathogens—A—bacteraemia indicating spots (eubacterial universal probes) not fluorescing, B Candida albicans probes, C Candida parapsilosis probes.

### DETAILED DESCRIPTION

### General

Provided herein are microarrays (protein and/or nucleic acid microarrays) containing of an array of spots on a solid substrate, wherein the spots are arranged to reduce the risk of array misalignment and/or to facilitate the visual interpretation of an array image by a human operator. Also provided herein are methods of using such arrays and kits containing such arrays.

Several types of low-density microarrays are known in the art, including array-in-tube format or array on shaft format, whereby the array is placed on a circular substrate (e.g., European Patent Application No. EP2305383, Liu et al., Clinical Chemistry 53:188-194 (2007), each of which is hereby incorporated by reference). Likewise, low-density arrays for pathogen detection containing several square sectors on a solid substrate such as a microscope slide are also known in the art (e.g. the Greiner Bio-One PapilloCheck array).

Such arrays may be read on commercially available microarray readers, resulting in graphical image file, such as .tif file, to be then evaluated by a standalone software. Such commercially available readers read the micro-arrays either from the side on which the probes are printed (e.g. Innopsys, Molecular Devices, Ditabis CheckScanner) or read through the substrate, resulting in a mirror image of the array (e.g. the Agilent array reader). Some readers, such as those manufactured by Tecan, can do both forms of array reading. Thus, assuring the array is interpreted in the correct orientation is an important aspect of pathogen detection by microarrays.

In certain embodiments, described herein are microarrays that reduce the risk of misorientation of the microarray or images of the microarray by containing always-detectable spots and never-detectable spots positioned on the array such that the array lacks both mirror and rotational symmetry.

In some embodiments, the microarrays described herein facilitate the visual interpretation of a microarray by a human operator by organizing the pathogen-specific spots of the array into one or more identification groups. For example, in some embodiments, such an identification group, the pathogen-specific spots may be specific for a related group of pathogens. Such a pathogen group can be, for example, taxonomically related and/or they can be medically related or be pathogens that are treated using the same or similar treatment methodology.

### DEFINITIONS

The articles “a” and “an” are used herein to refer to one or to more than one (i.e., to at least one) of the grammatical object of the article. By way of example, “an element” means one element or more than one element.

As used herein, the term “antibody” may refer to both an intact antibody and an antigen binding fragment thereof. Intact antibodies are glycoproteins that include at least two heavy (H) chains and two light (L) chains inter-connected by disulfide bonds. Each heavy chain includes a heavy chain variable region (abbreviated herein as V_{H}) and a heavy chain constant region. Each light chain includes a light chain variable region (abbreviated herein as V_{L}) and a light chain constant region. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The term “antibody” includes, for example, monoclonal antibodies, polyclonal antibodies, chimeric antibodies, humanized antibodies, human antibodies, multispecific antibodies (e.g., bispecific antibodies), single-chain antibodies and antigen-binding antibody fragments.

The term “control” includes any portion of an experimental system designed to demonstrate that the factor being tested is responsible for the observed effect, and is therefore useful to isolate and quantify the effect of one variable on a system.

As used herein, a “microarray” refers to a plurality of elements (e.g., spots), each immobilized on a solid surface of a substrate. Such spots can be, for example, always-detectable spots, (a detectable substance, such as a fluorescent molecule or an enzyme is immobilized at that position), can be never-detectable spots (no detectable substance or target-specific probe or antibody is immobilized at that position), pathogen-specific spots (a probe or antibody specific for a pathogen nucleic acid or protein is immobilized at that position) or a control spot (a probe or antibody specific for a control nucleic acid or protein is immobilized at that position).

The terms “polynucleotide,” oligonucleotide” and “nucleic acid” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified, such as by conjugation with a labeling component.

“Sample” refers to a solution that potentially contains pathogen nucleic acid or antibodies. A sample can be obtained, for example, from a subject, a culture, from potentially contaminated food or from an environmental source. If the sample is from a subject, the source of the sample may be solid tissue, as from a fresh, frozen and/or preserved organ, tissue sample, biopsy, or aspirate; blood or any blood constituents, serum, blood; bodily fluids such as cerebral spinal fluid, amniotic fluid, peritoneal fluid or interstitial fluid, urine, saliva, stool, tears; or cells from any time in gestation or development of the subject. The sample might be processed prior to analysis. For example, the sample may be cultured, lysed, and nucleic acids and/or proteins may be purified from other sample components using methods known in the art.

As used herein, the terms “subject” and “subjects” refer to an animal, e.g., a mammal including a non-primate (e.g., a cow, pig, horse, donkey, goat, camel, cat, dog, guinea pig, rat, mouse, sheep) and a primate (e.g., a monkey, such as a cynomolgous monkey, gorilla, chimpanzee and a human). In some embodiments, the subject may be a human adult, a human child, a human fetus, a human embryo and/or a human fertilized cell.

### Microarrays

Provided herein are microarrays (protein and/or nucleic acid microarrays) containing an array of spots on a solid substrate, wherein the spots are arranged to reduce the risk of array misalignment and/or to facilitate the visual interpretation of an array image by a human operator.

In certain embodiments, described herein are nucleic acid and/or protein microarrays containing an array of spots on a solid substrate. The spots on the solid substrate can be present in a regular pattern, such as a rectangular or square grid of spots. However, the distances between spots does not need to be uniform. For example, in some embodiments, the microarray is a rectangular grid of spots that is made up of multiple of sub-arrays of spots. In such embodiments the distance between adjacent sub-arrays can be different than the distance between adjacent spots within the sub-arrays. For example, in some embodiments the distance between adjacent sub-arrays is greater than the distance between adjacent spots within the sub-arrays. In some embodiments the distance between adjacent sub-arrays is about 1.1 times, 1.2 times, 1.3 times, 1.4 times, 1.5 times, 1.6 times, 1.7 times, 1.8 times, 1.9 times, 2.0 times, 2.5 times, 3 times, 4 times or 5 times the distance between spots within the sub-arrays.

In some embodiments, the array of spots includes a plurality of pathogen-specific spots and/or control spots. Such pathogen-specific spots and control spots can contain a nucleic acid probe or antibody immobilized on the solid substrate.

Many methods for immobilizing nucleic acids and antibodies on a variety of solid surfaces are known in the art. For instance, the solid surface may be a membrane, glass or plastic. The nucleic acid or antibody may be covalently bound or noncovalently attached through nonspecific binding.

A wide variety of organic and inorganic polymers, as well as other materials, both natural and synthetic, may be employed as the material for the solid surface. Illustrative solid surfaces include nitrocellulose, nylon, glass, diazotized membranes (paper or nylon), silicones, polyformaldehyde, cellulose, and cellulose acetate. In addition, plastics such as polyethylene, polypropylene, polystyrene, and the like can be used. Other materials which may be employed include paper, ceramics, metals, metalloids, semiconductive materials, cermets or the like. In addition substances that form gels can be used. Such materials include proteins (e.g., gelatins), lipopolysaccharides, silicates, agarose and polyacrylamides. Where the solid surface is porous, various pore sizes may be employed depending upon the nature of the system.

In preparing the surface, a plurality of different materials may be employed, particularly as laminates, to obtain various properties. For example, proteins (e.g., bovine serum albumin) or mixtures of macromolecules (e.g., Denhardt's solution) can be employed to avoid non-specific binding, simplify covalent conjugation, enhance signal detection or the like.

If covalent bonding between a compound and the surface is desired, the surface will usually be polyfunctional or be capable of being polyfunctionalized. Functional groups which may be present on the surface and used for linking can include carboxylic acids, aldehydes, amino groups, cyano groups, ethylenic groups, hydroxyl groups, mercapto groups, epoxy, and the like. The manner of linking a wide variety of compounds to various surfaces is well known and is amply illustrated in the literature. For example, methods for immobilizing nucleic acids by introduction of various functional groups to the molecules is known (see, e.g., Bischoff et al., Anal. Biochem. 164:336-344 (1987); Kremsky et al., Nuc. Acids Res. 15:2891-2910 (1987)). Modified nucleotides can be placed on the target using PCR primers containing the modified nucleotide, or by enzymatic end labeling with modified nucleotides.

There are many possible approaches to coupling nucleic acids and/or antibodies to glass that employ commercially available reagents. For instance, materials for preparation of silanized glass with a number of functional groups are commercially available or can be prepared using standard techniques.

The nucleic acids and antibodies can also be immobilized on other surfaces. For instance, biotin labeled nucleic acids and antibodies can be bound to commercially available avidin-coated surfaces. Streptavidin or anti-digoxigenin antibody can also be attached to silanized glass slides by protein-mediated coupling using e.g., protein A following standard protocols (see, e.g., Smith et al. Science, 258:1122-1126 (1992; incorporated by reference herein)). Biotin or digoxigenin end-labeled nucleic acids can be prepared according to standard techniques.

Additional methods for immobilizing nucleic acids and/or antibodies to a solid substrate are described in U.S. Pat. Nos. 5,143,854, 5,445,934, 5,830,645, 6,815,078, 7,667,194, 7,713,749, 8,014,577, and 8,263,532, each of which is incorporated by reference.

In some embodiments, the array of spots includes one or more always-detectable spots containing a detectable substance immobilized to the solid substrate (e.g., an always-fluorescing spot containing a fluorescent dye immobilized on the solid substrate). The detectable substance can contain any material having a detectable physical or chemical property. Such detectable labels are well known in the art. Thus a label is any composition detectable by spectroscopic, photochemical, biochemical, immunochemical, electrical, optical or chemical means. Useful detectable substances in microarrays described herein include fluorescent dyes (e.g., fluorescein isothiocyanate, Texas red, rhodamine, and the like) radiolabels (e.g., ³H, ¹²⁵I, ³⁵S, ¹⁴C, or ³²P), enzymes (e.g., horse radish peroxidase, alkaline phosphatase and others commonly used in an ELISA).

In some embodiments, the array of spots includes one or more never-detectable spots. Such spots, for example, may be empty positions in the array or they may be spotted with spotting buffer that does not contain a detectable substance, a nucleic acid probe or an antibody (e.g., never-fluorescing spots containing neither a fluorescent dye nor a nucleic acid probe immobilized to the solid substrate).

In certain embodiments, the one or more always-detectable spots and the one or more never-detectable spots are positioned such that the array of spots has neither rotational symmetry nor mirror symmetry. For example, in some embodiments the position of one or more always-detectable spots and one or more never-detectable spots are such that rotation of the microarray by 90 degrees, 180 degrees or 270 degrees results in at least one always-detectable spot being in a position occupied by a never-detectable spot in an un-rotated array. In another embodiment, the position of one or more always-detectable spots and one or more never-detectable spots are such that flipping the microarray on its horizontal or vertical axis results in at least one always-detectable spot being in a position occupied by a never-detectable spot in an un-rotated array. In some embodiments the position of one or more always-detectable spots and one or more never-detectable spots are such that rotation of the microarray by 90 degrees, 180 degrees or 270 degrees and flipping the microarray on its horizontal or vertical axis results in at least one always-detectable spot being in a position occupied by a never-detectable spot in an un-rotated array. In certain embodiments the position of one or more always-detectable spots and one or more never-detectable spots are such that rotation of the microarray by 90 degrees, 180 degrees or 270 degrees results in at least one never-detectable spot being in a position occupied by an always-detectable spot in an un-rotated array. In some embodiments the position of one or more always-detectable spots and one or more never-detectable spots are such that flipping the microarray on its horizontal or vertical axis results in at least one never-detectable spot being in a position occupied by an always-detectable spot in an un-rotated array. In certain embodiments the position of one or more always-detectable spots and one or more never-detectable spots are such that rotation of the microarray by 90 degrees, 180 degrees or 270 degrees and flipping the microarray on its horizontal or vertical axis results in at least one never-detectable spot being in a position occupied by an always-detectable spot in an un-rotated array.

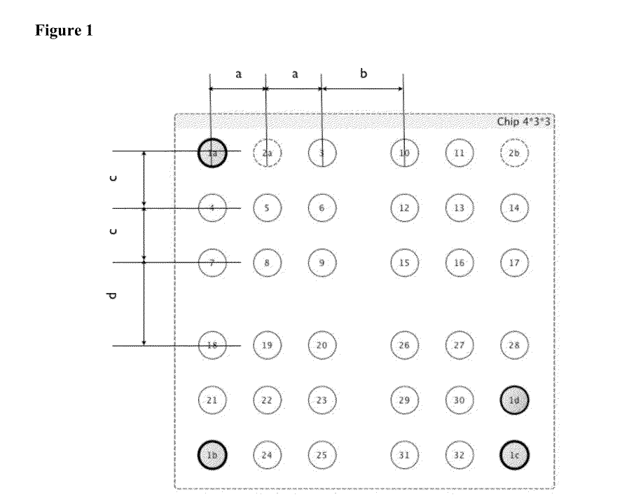

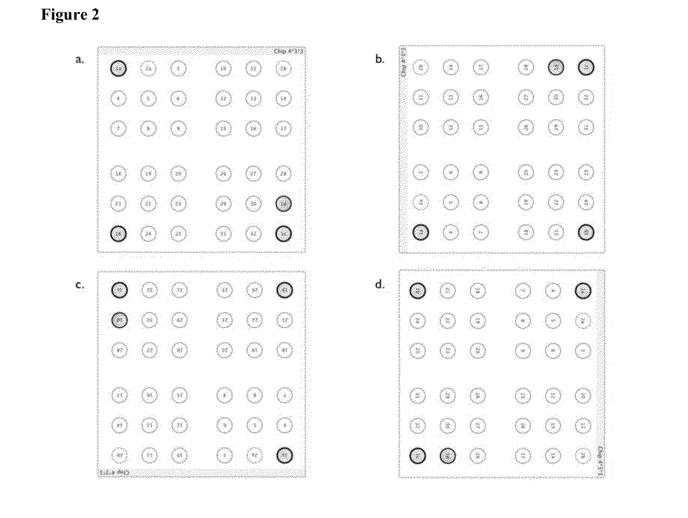

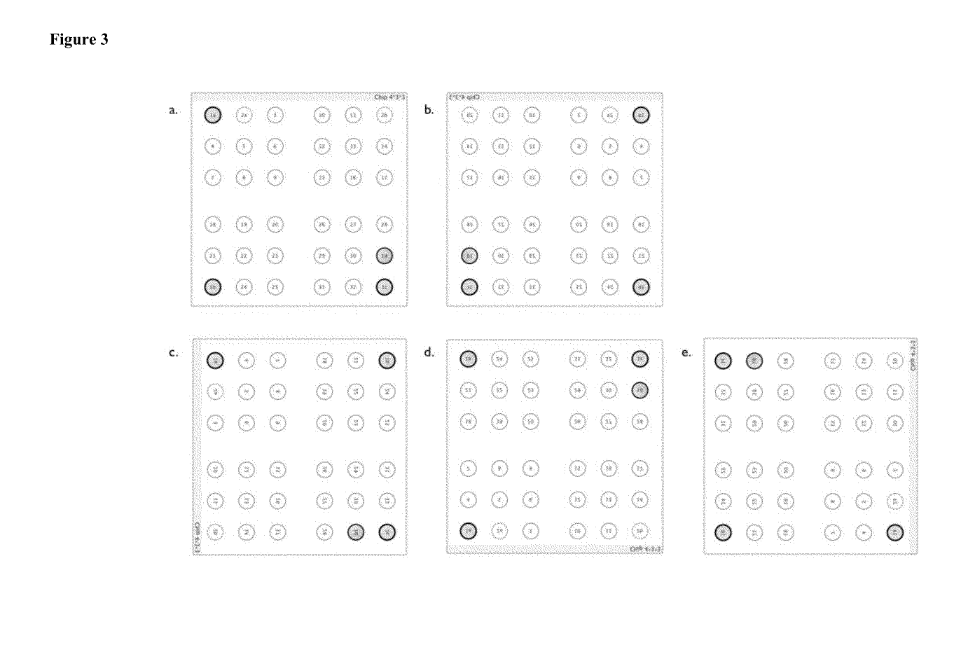

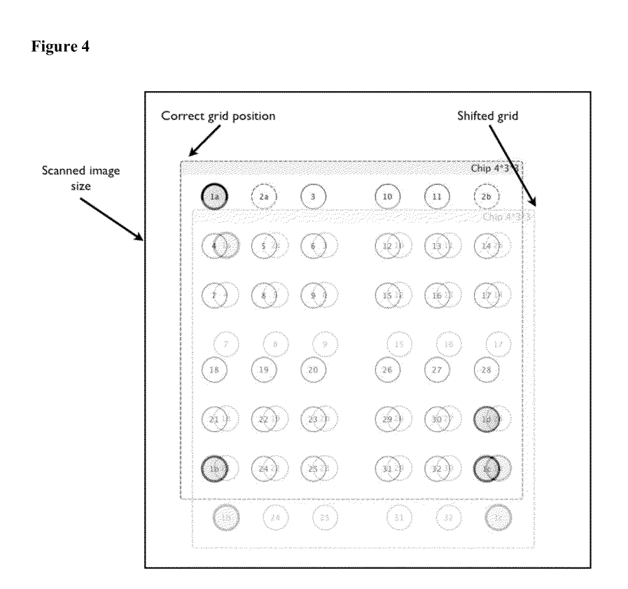

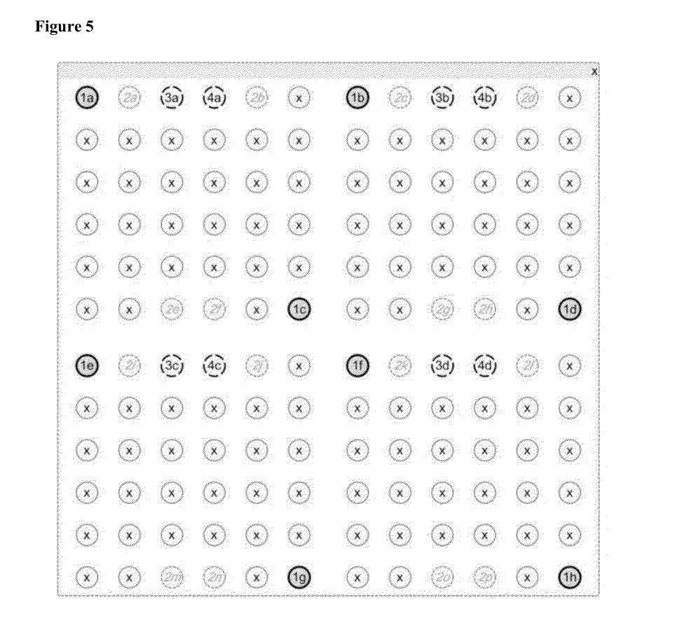

An exemplary array is provided in FIG. 1. This array includes four sub-arrays of three times three spots, the distance between the rows or columns within the segment (c, a) is different from that between the adjacent sectors (d or b). The array also includes four always-fluorescing spots (always positive, 1a through 1d) and two never-fluorescing (never positive, 2a and 2b) spots. Spots 3 to 32 are target selective probes. As depicted in FIG. 2, such an array lacks rotational symmetry. As depicted in FIG. 3, such an array also lacks mirror-image symmetry. As depicted in FIG. 4, inadvertently shifting the array by one row from the correct position results in pivotal elements 1b and 1d not being detected, allowing the alignment to be rejected.

In some embodiments, the microarray contains a rectangular grid of spots (e.g., a square grid of spots), and an always-fluorescing spot is positioned in at least one corner of the rectangular grid of spots. In some embodiments, always-fluorescing spots are positioned at 2 or 3 corners of the rectangular grid of spots and a never-fluorescing spot is positioned in the other corners of the rectangular grid of spots. In some embodiments, one never-detectable spots is positioned in one corner of a sub-array and four always-detectable elements are positioned in the remaining three corners of the sub-arrays in order to facilitate the positioning of the reading grid and on the edge of the sub-array connecting one of the always-detectable spots with the never-detectable spot next to the always-detectable spot. (e.g., as depicted in FIG. 1). One more never-detectable spot is positioned on the edge connecting the other always-detectable spot with the never-detectable spot, next to the always-detectable spot. In some embodiments, the array includes two always-detectable spots, four never-detectable spots and two control spots, the always-detectable spots being placed diagonally at the corners of the array, two of the never-detectable spots being placed on the edge of the array next to the always-detectable spots in an arrangement never-detectable/control/control/never-detectable/and two never-detectable spots are placed on the parallel edge opposite to the control spots.

In some embodiments, the layout of the pathogen-specific spots on the microarrays described herein are suitable for visual evaluation without the use of computers. In some embodiments, the microarray contains a plurality of pathogen-specific spots that are organized as one or more identification groups, each group containing probes or antibodies reacting to nucleic acids and/or proteins from a related group of pathogens. In some embodiments, the identification group is a group of taxonomically related pathogens. In some embodiments, the identification group is a group of medically or treatment options related pathogens.

For example, in some embodiments, in such an identification group, the pathogen-specific nucleic acid probe or antibody contained by each spot within an identification group is specific for a target nucleic acid or protein from a related group of pathogens. In some embodiments, the identification groups contain at least 2, 3, 4, 5, 6, 7 or 8 spots arranged in a square, rectangle and/or line. In some embodiments, the identification groups contain no more than 4, 5, 6, 7, 8, 9, 10, 11, 12, 13, 14, 15, 16, 17, 18, 19 or 20 spots arranged in a square, rectangle and/or line. In some embodiments the related group of pathogens contains no more than 30, 29, 28, 27, 26, 25, 24, 23, 22, 21, 20, 19, 18, 17, 16, 15, 14, 13, 12, 11, 10, 9, 8, 7, 6, 5, 4, 3 or 2 pathogens.

In some embodiments, provided herein is a kit comprising a microarray described herein. In some embodiments the kit includes a microarray pattern identification aid, such as a rotary dial device and/or a printed pattern identification tree. In some embodiments the kit also includes instructions for using the microarray device.

### Methods

In some embodiments, provided herein are methods of performing a nucleic acid microarray analysis using a microarray described herein above. The methods can be used, for example, for diagnosis or prognosis of a subject and/or for detection of food or environmental contamination.

In some embodiments, the method includes the step of contacting a sample with the microarray. The sample can be obtained, for example, from a patient, from a non-human animal, from a cell or bacterial culture, from a food source and/or from the environment (e.g., an air or water sample).

In some embodiments, the sample contains nucleic acids and/or proteins. In certain embodiments the sample will be processed before it is contacted with the microarray. For example, pathogens in the sample can be cultured, cells in the sample can be lysed and/or components of the sample (e.g., nucleic acids and/or proteins) can be purified prior to contacting the sample with the microarray. In some embodiments, the method includes performing an amplification procedure (e.g., a PCR procedure) on a nucleic acid of the sample before or after contacting it with the microarray.

In some embodiments, the nucleic acids and/or proteins in the sample are labeled with a detectable label. The nucleic acids and proteins used in the methods described herein may be detectably labeled prior to contacting the sample with the microarray. Alternatively, a detectable label may be selected which binds to the nucleic acids and/or proteins after they are immobilized on the microarray.

Any label or detectable group attached to the probe nucleic acids or proteins can be used in the methods described herein, so long as it does not significantly interfere with the hybridization of the probe to the target sequence or the binding of the antibody to the protein. The detectable group can be any material having a detectable physical or chemical property. Such detectable labels are well known in the art. Thus a label can be any composition detectable by spectroscopic, photochemical, biochemical, immunochemical, electrical, optical or chemical means. Useful detectable substances in methods described herein include fluorescent dyes (e.g., fluorescein isothiocyanate, Texas red, rhodamine, and the like) radiolabels (e.g., ³H, ¹²⁵I, ³⁵S, ¹⁴C, or ³²P), enzymes (e.g., horse radish peroxidase, alkaline phosphatase and others commonly used in an ELISA).

The nucleic acids and proteins can be indirectly labeled using ligands for which detectable anti-ligands are available. For example, biotinylated nucleic acids and proteins can be detected using labeled avidin or streptavidin according to techniques well known in the art. In addition, antigenic or haptenic molecules can be detected using labeled antisera or monoclonal antibodies. For example, N-acetoxy-N-2-acetylaminofluorene-labelled or digoxigenin-labeled probes can be detected using antibodies specifically immunoreactive with these compounds (e.g., FITC-labeled sheep anti-digoxigenin antibody (Boehringer Mannheim)). In addition, labeled antibodies to thymidine-thymidine dimers can be used (Nakane et al. ACTA Histochem. Cytochem. 20:229 (1987), incorporated herein by reference).

Generally, labels which are detectable in as low a copy number as possible, thereby maximizing the sensitivity of the assay, and yet be detectable above any background signal are preferred. A label is preferably chosen that provides a localized signal, thereby providing spatial resolution of the signal from each target element. The labels may be coupled to the nucleic acids and proteins in a variety of means known to those of skill in the art.

In some embodiments, the method includes the step of incubating the microarray under conditions that would permit target proteins and/or target nucleic acids in the sample, if present, to become immobilized on spots of the microarray containing nucleic acid probes or antibodies specific for such a target nucleic acids or proteins. Such conditions are well known in the art and are described, in, for example, U.S. Pat. Nos. 5,143,854, 5,445,934, 5,830,645, 6,815,078, 7,667,194, 7,713,749, 8,014,577, and 8,263,532, each of which is incorporated by reference. In some embodiments the microarray is washed one or more times to remove non-immobilized nucleic acids and/or proteins.

In some embodiments, the method includes the step of detecting the presence of proteins or nucleic acids from the sample immobilized on at least one of the spots of the microarray. In some embodiments, the method includes the step of detecting fluorescence emitted by the spots of the microarray. In some embodiments the method includes the step of contacting the microarray with a chromogenic substrate and detecting a color change of the spots of the microarray. In some embodiments the method includes the step of generating an image of the microarray during the detection step.

Standard methods for detection and analysis of signals generated by detectable labels can be used and the particular methods will depend upon which labels are used in the method. Thus, when fluorescent labels are used, the microarray can be imaged using a fluorescence microscope with a polychromatic beam-splitter to avoid color-dependent image shifts. The different color images can be acquired with a CCD camera and the digitized images stored in a computer.

In certain embodiments, the method includes the step of visually interpreting the image of the microarray. In some embodiments the step of visually interpreting the image of the microarray is performed by the operator without the aid of image recognition software.

### EXAMPLES

### Example 1

### Microarray Spotting

Sixteen-well glass, epoxy modified substrates (Nexterion® Slide E MPX) were spotted with solutions of amino-modified fluorescent dye (“always on” spots), 5′-amino modified amplification control oligonucleotide probes (universal bacterial 16S rDNA probes, “amplification control” spots), 5′-amino modified pathogen-specific oligonucleotide probes (25-30 nt long probes, “target-specific” spots) and spotting buffer alone (“always off” spots). The spotting buffer was prepared by combining 99 parts of a Nexterion® Spot Solution with 1 part of a Nexterion® Sarcosyl Solution.

TABLE 1
Amplification Control Probe Sequences

| Probe | Sequence (5′ to 3′) |
|---|---|
| 3(x) Uni-Euba-I | AACAGGATTAGATACCCTGGTAGTCCACGC |
| 4(x) Uni-Euba-II | GGGACCCGCACAAGCGGTGGAGCAT |

The spotted arrays were left to react for 30 minutes at room temperature and 90% relative humidity, then dried and heated for 1 hr. to 100° C. The arrays were then washed and blocked according to the Nexterion® protocol (Nexterion®® Slide E MPX 16, DNA-application, Document No.: LS6-HBM-M-002, Version: 1.2, Schott AG, April 2009, incorporated by reference). The slides were dried by subjecting them to a stream of dry, clean air and stored at room temperature, protected from light and humidity.

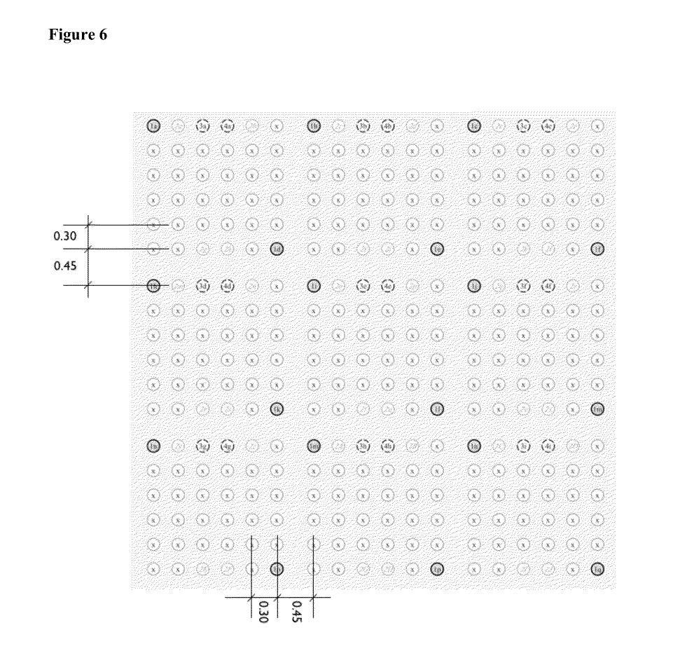

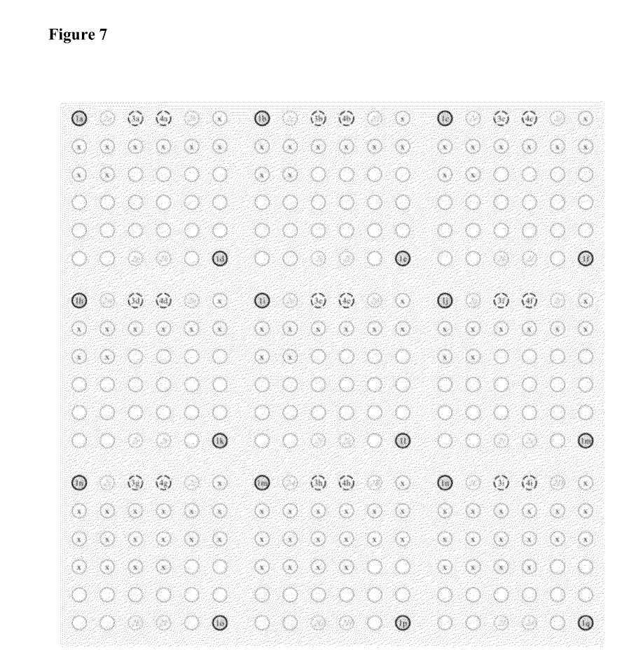

The dimensions of the array and positions of the spots are provided in FIG. 6, with elements 1(a-q) representing “always on” pivotal spots, elements 2(a-z and A-J) representing “always off” pivotal spots, and elements 3(a-i) and 4(a-i) representing “amplification control spots.” As depicted in FIG. 7, target-specific probes were printed only positions denoted with “x”, other positions were left empty.

### Example 2

### DNA Amplification and Microarray Hybridization

Mixed bacterial 16S rDNA standard was amplified by PCR. The PCR master mix is provided in Table 2. The PCR reaction was performed using dye labelled dUTP and universal 16S rDNA primers F8 and R (sequences provided in Table 3, PCR-a). A mixture of plasmids containing plasmid DNA encoding mecA and blaZ genes was also amplified under the same conditions, using the mecA and blaZ primer pairs (sequences provided in Table 3, PCR-b). The PCR conditions used for both reactions are provided in Table 4.

TABLE 2
PCR Master Mix

| Component | Volume per 1rxn (25 microliters) |
|---|---|
| Fermentas PCR Master Mix | 12.5 |
| (2X) # K0171 | |
| Dyomics Dy547-dUTP | 5 |
| Water for molecular biology | 10 |
| (Template) | (2) |

TABLE 3
Primers

| Use/Name | Sequence (5′ to 3′) |
|---|---|
| 16S rDNA F8 | AGA GTT TGA TCC TGG CTC AG |
| 16S rDNA R1409 | GGC CTT GTA CAC ACC GCC CGT CA |
| BlaZ F | CAA CAT TTC CGT GTC GCC CTT |
| BlaZ R | ATC GTG GTG TCA CGC TCG |
| MecA F | CGG TAA CAT TGA TCG CAA CG |
| MecA R | CGT TGT AAC CAC CCC AAG AT |

TABLE 4
PCR Conditions

| | |
|---|---|
| | 95° C. 3 min |
| 40x | 95° C. 15 s |
| | 60° C. 15 s |
| | 72° C. 60 s |
| | 72° C. 2 min |
| | 4° C. ∞ |

Hybridisation buffer was prepared by mixing one volume of Nexterion® Oligo Hyb Buffer, SCHOTT Technical Glass Solution GmbH #1116890, with three volumes of Nexterion® Hyb Buffer, SCHOTT Technical Glass Solution GmbH #1066075. Equal volumes of PCR-a and PCR-b products were mixed and 5 microliters of the mixture were diluted with 30 microliters of hybridisation buffer. The microarray was mounted into the Nexterion® IC-16 reusable incubation chamber (order code: 1262705) and pre-heated to 70° C. for 15 minutes in an Eppendorf Comfort mixer/heater. While still on a heating block, 30 microliters of the DNA solutions were added to individual wells, the incubation chamber was sealed with a length of adhesive tape and the microarrays were hybridized by mixing (450 rpm) for 4 minutes at 70° C., then at 37° C. for a further 30 minutes. Following the hybridisation, the chamber was opened, and the array was washed twice with 200 microliters of a washing solution (2×SSC containing 0.2% SDS). Then, the array was taken out from the incubation chamber and washed twice with 50 millilitres of the washing solution, then twice with 2×SSC and finally twice with 0.2×SSC. The microarrays were dried with a stream of clean air, and stored at room temperature protected from light and humidity until scanned.

The arrays were scanned using a Tecan Reloaded scanner at a resolution of 10 micrometres per pixel and Cy-3 laser/filter settings. Raw images were saved as .tiff files for further processing.

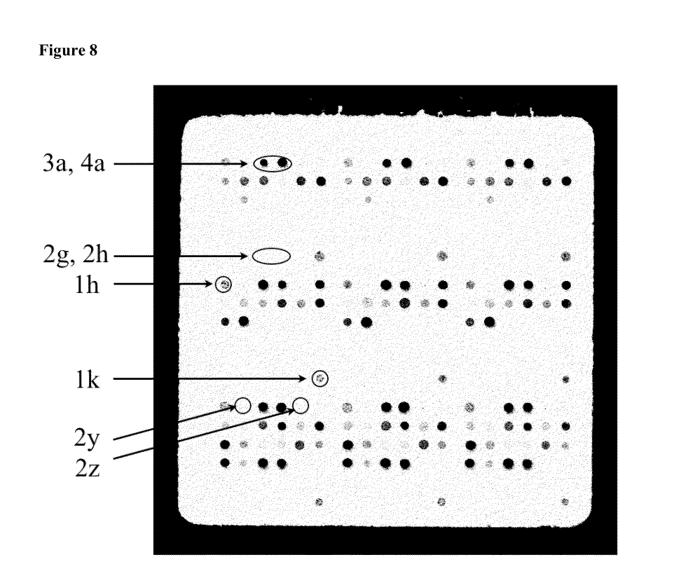

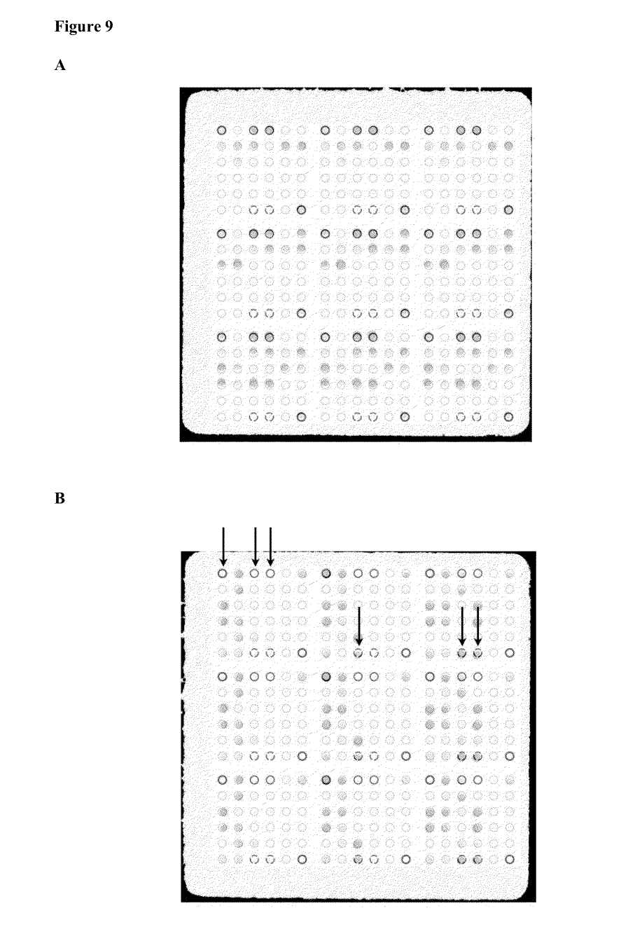

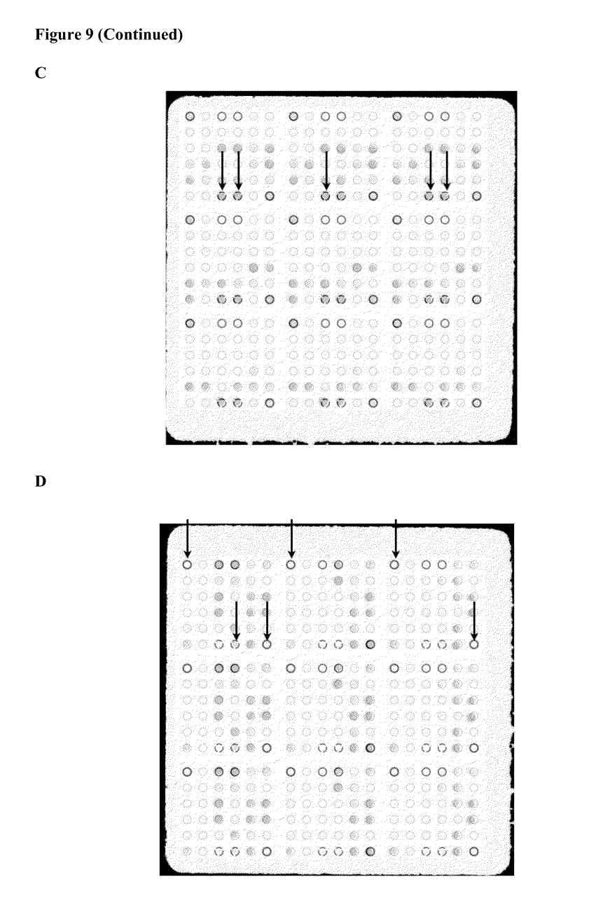

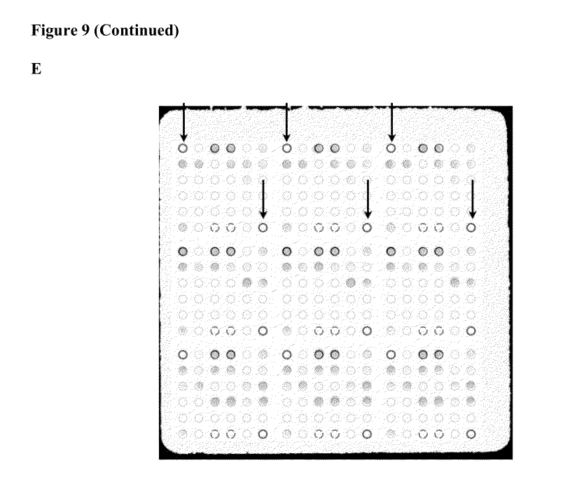

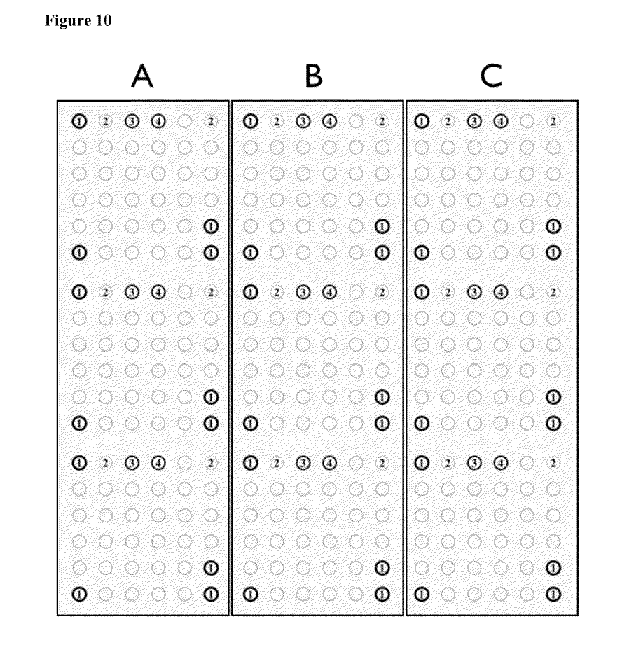

An exemplary image of the microarray is provided in FIG. 8. As depicted in FIG. 9, based on the position of the “always on” and “always off” spots in the chip, it is possible to determine whether the scan is properly oriented (FIG. 9a), the scan is rotated 90 degrees (FIG. 9b), the scan is rotated 180 degrees (FIG. 9c), the scan is rotated 270 degrees (FIG. 9d), or the scan is flipped horizontally (FIG. 9e).

### Example 3

### Microarray Spotting

Sixteen-well glass, epoxy modified substrates (Nexterion® Slide E MPX) are spotted with amino-modified fluorescent dye (“always on” spots), 5′-amino modified amplification control oligonucleotide probes (universal bacterial 16S rDNA probes) (“amplification control” spots), and a selection of 5′-amino modified multispecific, group specific or specific oligonucleotide probes (“target-specific” spots, probe sequences provided in Table 5), or a spotting buffer alone (“always off” spots). The spotting buffer is prepared by combining 99 parts of the Nexterion® Spot Solution with 1 part of the Nexterion® Sarcosyl Solution. The concentration of individual components in the spotting buffer is 30 micromoles/Litre.

TABLE 5
Probe sequences

| Name | Sequence [5′-3′] |
|---|---|
| aba1 | CAAGCTACCTTCCCCCGCT |
| aba2 | GTAACGTCCACTATCTCTAGGTATTAACTAAAGTAG |
| aba4 | GCAGTATCCTTAAAGTTCCCATCCGAAAT |
| ajo2 | TCCCAGTATCGAATGCAATTCCTAAGTT |
| ajo3 | GAAAGTTCTTACTATGTCAAGACCAGGTAAG |
| ajo4 | CTTAACCCGCTGGCAAATAAGGAAAA |
| alw1 | GAGATGTTGTCCCCCACTAATAGGC |
| alw2 | TGACTTAATTGGCCACCTACGCG |
| alw3 | CCCATACTCTAGCCAACCAGTATCG |
| ara1 | CGCTGAATCCAGTAGCAAGCTAC |
| ara2 | GTCCACTATCCTAAAGTATTAATCTAGGTAGCCT |
| ara3 | CCGAAGTGCTGGCAAATAAGGAAA |
| cif1 | GCTCCTCTGCTACCGTTCG |
| cif2 | CCACAACGCCTTCCTCCTCG |
| cif3 | TCTGCGAGTAACGTCAATCGCTG |
| cik1 | CGGGTAACGTCAATTGCTGTGG |
| cik2 | CGAGACTCAAGCCTGCCAGTAT |
| ecl4 | GCGGGTAACGTCAATTGCTGC |
| ecl6 | CTACAAGACTCCAGCCTGCCA |
| ecl7 | TACCCCCCTCTACAAGACTCCA |
| ena2 | GGTTATTAACCTTAACGCCTTCCTCCT |
| ena3 | CAATCGCCAAGGTTATTAACCTTAACGC |
| ena4 | TCTGCGAGTAACGTCAATCGCC |
| kpn1 | GCTCTCTGTGCTACCGCTCG |
| kpn2 | GCATGAGGCCCGAAGGTC |
| klo1 | TCGTCACCCGAGAGCAAGC |
| klo2 | CCAGCCTGCCAGTTTCGAATG |
| eco2 | GTAACGTCAATGAGCAAAGGTATTAACTTTACTCCCTTCC |
| eco3 | CCGAAGGCACATTCTCATCTCTGAAAACTTCCGTGGATG |
| mom2 | GCCATCAGGCAGATCCCCATAC |
| mom3 | CTTGACACCTTCCTCCCGACT |
| mom4 | CATCTGACTCAATCAACCGCCTG |
| pmi3 | GTCAGCCTTTACCCCACCTACTAG |
| pmi4 | GGGTATTAACCTTATCACCTTCCTCCC |
| pmi5 | CCAACCAGTTTCAGATGCAATTCCC |
| pmi6 | GTTCAAGACCACAACCTCTAAATCGAC |
| pvu2 | CTGCTTTGGTCCGTAGACGTCA |
| pvu4 | TTCCCGAAGGCACTCCTCTATCTCTA |
| psa4 | GATTTCACATCCAACTTGCTGAACCA |
| psa5 | TCTCCTTAGAGTGCCCACCCG |
| psa6 | CGTGGTAACCGTCCCCCTTG |
| sem1 | CTCCCCTGTGCTACCGCTC |
| sem2 | CACCACCTTCCTCCTCGCTG |
| sem3 | GAGTAACGTCAATTGATGAGCGTATTAAGC |
| sma1 | AGCTGCCTTCGCCATGGATGTTC |
| sma3 | TGGGATTGGCTTACCGTCGC |
| spn1 | CTCCTCCTTCAGCGTTCTACTTGC |
| spn3 | GGTCCATCTGGTAGTGATGCAAGTG |
| spn5 | TCTTGCACTCAAGTTAAACAGTTTCCAAAG |
| spy1 | ATTACTAACATGCGTTAGTCTCTCTTATGCG |
| spy2 | CTGGTTAGTTACCGTCACTTGGTGG |
| spy3 | TTCTCCAGTTTCCAAAGCGTACATTG |
| efa1 | CAAGCTCCGGTGGAAAAAGAAGC |
| efa2 | CATCCATCAGCGACACCCGA |
| efa3 | ACTTCGCAACTCGTTGTACTTCCC |
| efa42 | CCGTCAAGGGATGAACAGTTACTCTCATCCTTGTTCTTC |
| efa43 | ATTAGCTTAGCCTCGCGACTTCGCAACTCGTTGTACTTC |
| efa51 | CTCCGGTGGAAAAAGAAGCGT |
| efa52 | CTCCCGGTGGAGCAAG |
| sta1 | CTCTATCTCTAGAGCGGTCAAAGGAT |
| sta2 | CAGTCAACCTAGAGTGCCCAACT |
| sta3 | AGCTGCCCTTTGTATTGTCCATT |
| sta4 | ATGGGATTTGCATGACCTCGCG |
| sar1 | CCGTCTTTCACTTTTGAACCATGC |
| sar2 | AGCTAATGCAGCGCGGATC |
| sar3 | TGCACAGTTACTTACACATATGTTCTT |
| sep1 | AAGGGGAAAACTCTATCTCTAGAGGG |
| sep2 | GGGTCAGAGGATGTCAAGATTTGG |
| sep3 | ATCTCTAGAGGGGTCAGAGGATGT |
| efc1 | CCACTCCTCTTTCCAATTGAGTGCA |
| efc2 | GCCATGCGGCATAAACTGTTATGC |
| efc3 | CCCGAAAGCGCCTTTCACTCTT |
| efc4 | GGACGTTCAGTTACTAACGTCCTTG |
| cal1 | CCAGCGAGTATAAGCCTTGGCC |
| cpa1 | TAGCCTTTTTGGCGAACCAGG |
| uni1 | AACAGGATTAGATACCCTGGTAGTCCACGC |
| uni2 | GGGACCCGCACAAGCGGTGGAGCAT |

The spotted arrays are left to react for 30 minutes at room temperature and 90% relative humidity, then dried and heated for 1 hr. to 100° C. The arrays are then washed and blocked according to the Nexterion® protocol (Nexterion®® Slide E MPX 16, DNA-application, Document No.: LS6-HBM-M-002, Version: 1.2, Schott AG, April 2009, incorporated by reference). The slides are dried using a stream of dry clean air and stored at room temperature, protected from light and humidity.

### Example 4

### DNA Amplification and Microarray Hybridization

Individual bacterial 16S rDNA standards were amplified by PCR using master mix (Table 6) containing dye labelled dUTP and universal 16S rDNA primers F8 and R 1409 (Table 7) using the PCR conditions set forth in Table 8.

TABLE 6
Master mix composition (volume of reaction 25 microliters)

| Component | Volume per 1rxn (25 microliters) |
|---|---|
| Fermentas PCR Master Mix | 12.5 |
| (2X) # K0171 | |
| Dyomics Dy547-dUTP | 5 |
| Water for molecular biology | 10 |
| (Template) | (2) |

TABLE 7
Primers

| Use/Name | Sequence (5′ to 3′) |
|---|---|
| 16S rDNA F8 | AGA GTT TGA TCC TGG CTC AG |
| 16S rDNA R1409 | GGC CTT GTA CAC ACC GCC CGT CA |

TABLE 8
PCR Conditions

| | |
|---|---|
| | 95° C. 3 min |
| 40x | 95° C. 15 s |
| | 60° C. 15 s |
| | 72° C. 60 s |
| | 72° C. 2 min |
| | 4° C. ∞ |

Hybridisation buffer was prepared by mixing one volume of Nexterion® Oligo Hyb Buffer, SCHOTT Technical Glass Solution GmbH #1116890, with three volumes of Nexterion® Hyb Buffer, SCHOTT Technical Glass Solution GmbH #1066075. Equal volumes of PCR-a and PCR-b products were mixed and 5 microliters of the mixture were diluted with 30 microliters of hybridisation buffer. The microarray was mounted into the Nexterion® IC-16 reusable incubation chamber (order code: 1262705) and pre-heated to 70° C. for 15 minutes in an Eppendorf Comfort mixer/heater. While still on a heating block, 30 microliters of the DNA solutions were added to individual wells, the incubation chamber was sealed with a length of adhesive tape and hybridised by mixing (450 rpm) for 4 minutes at 70° C., followed by mixing at 37° C. for a further 30 minutes. Following the hybridization, the chamber was opened and the array was washed twice with 200 microliters of a washing solution (2×SSC containing 0.2% SDS). Then, the array was taken out from the incubation chamber and washed twice with 50 millilitres of the washing solution, then twice with 2×SSC and finally twice with 0.2×SSC. The microarrays were dried with a stream of clean air and stored at room temperature protected from light and humidity until scanned.

The arrays were scanned by Tecan Reloaded scanner at a resolution of 10 micrometres per pixel and Cy-3 laser/filter settings. Raw images were saved as .tiff files for further processing. The prepared microarrays are capable of detecting pathogens listed in Table 9.

TABLE 9
Pathogens Detected

| Group | Subgroup | Species |
|---|---|---|
| Enteric rods | | Citrobacter freundii |
| Enteric rods | | Citrobacter koseri |
| Enteric rods | | Enterobacter aerogenes |
| Enteric rods | | Enterobacter cloacae |
| Enteric rods | | Escherichia coli |
| Enteric rods | | Klebsiella oxytoca |
| Enteric rods | | Klebsiella pneumoniae |
| Enteric rods | | Morganella morganii |
| Enteric rods | | Proteus mirabilis |
| Enteric rods | | Proteus vulgaris |
| Enteric rods | | Serratia marcescens |
| Enterococci | Group D | Enterococcus faecalis |
| | streptococci | |
| Enterococci | Group D | Enterococcus faecium |
| | streptococci | |
| Gram negative | | Acinetobacter baumannii |
| aerobic cocci | | |
| Gram negative | | Acinetobacter johnsonii |
| aerobic cocci | | |
| Gram negative | | Acinetobacter lwoffii |
| aerobic cocci | | |
| Gram negative | | Acinetobacter radioresistens |
| aerobic cocci | | |
| Gram negative | | Pseudomonas aeruginosa |
| aerobic rods | | |
| Gram negative | | Stenotrophomonas maltophilia |
| aerobic rods | | |
| Staphylococci | Coagulase positive | Staphylococcus aureus |
| Staphylococci | Coagulase negative | Staphylococcus epidermidis |
| Streptococci | Alpha haemolytic | Streptococcus pneumoniae |
| Streptococci | Group A | Streptococcus pyogenes |
| | Streptococci | |
| Fungi | Candida | Candida albicans |
| Fungi | Candida | Candida parapsilosis |

### Example 5

### Microarray Design with Square Identification Groups

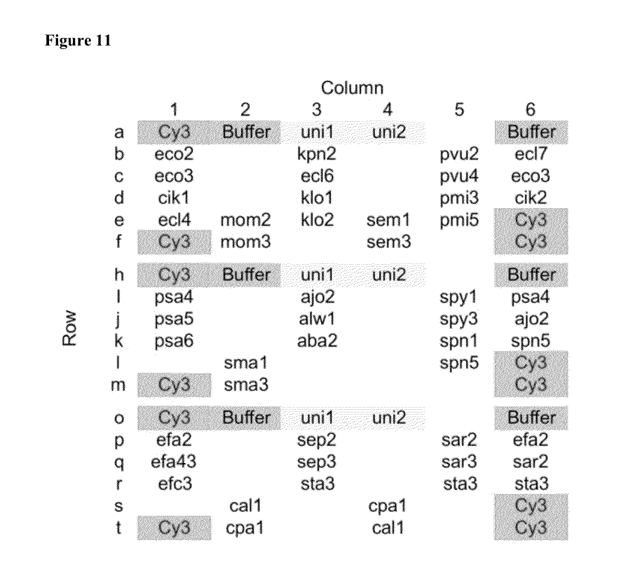

A microarray is printed according the procedure set forth in Example 3, using the probe layout according to FIG. 11 (only one of the three identical sub-arrays is shown).

The pathogen DNA is amplified and the micro-array is hybridised according to the procedure of Example 4.

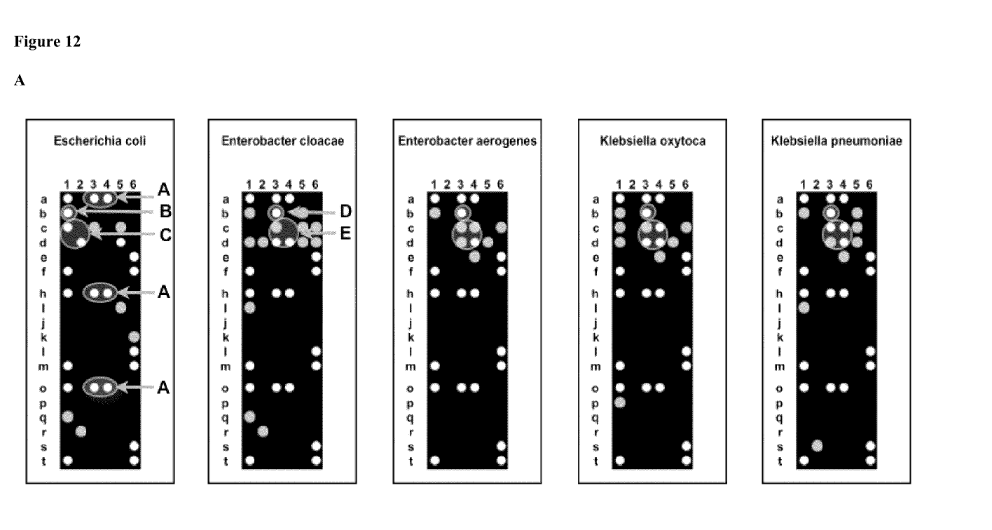

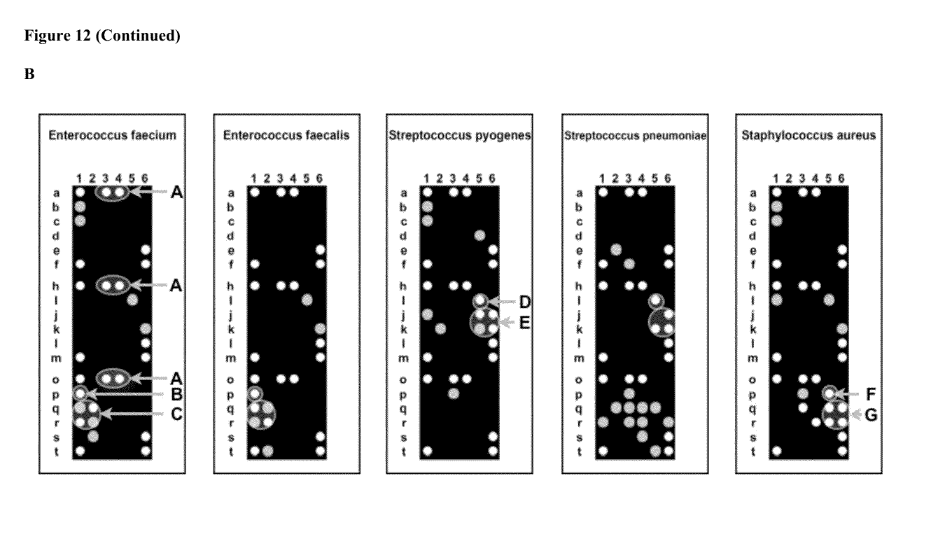

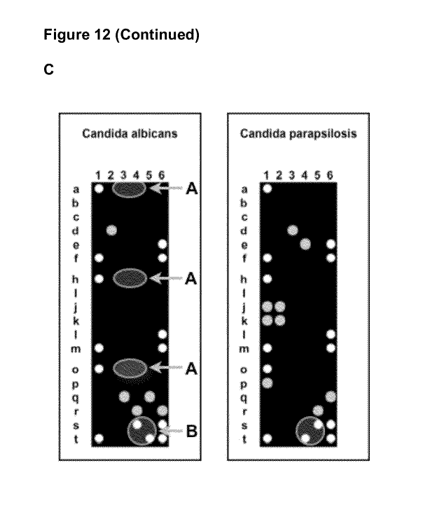

The micro-array is visually evaluated and the light output of each spot is recorded as no-fluorescence, weak fluorescence or strong fluorescence and transferred to a graphical form. Representative graphical presentations are depicted in FIG. 12.

### Example 6

### Microarray Design with Linear Identification Groups

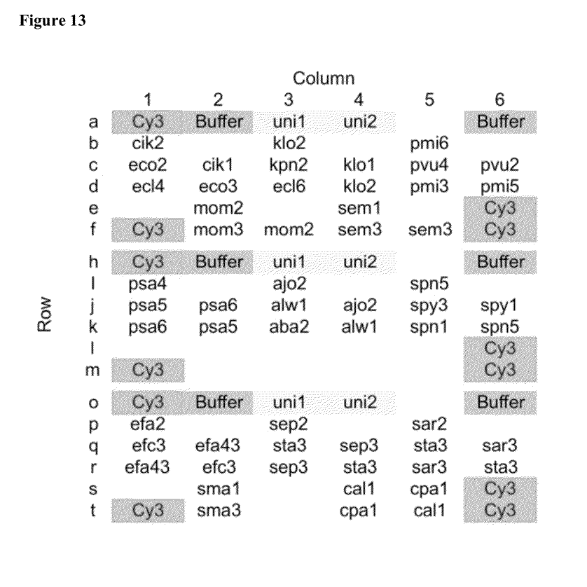

A microarray is printed according the procedure set forth in Example 3, using the probe layout according to FIG. 13 (only one of the three identical sub-arrays is shown).

The pathogen DNA is amplified and the micro-array is hybridised according to the procedure of Example 4.

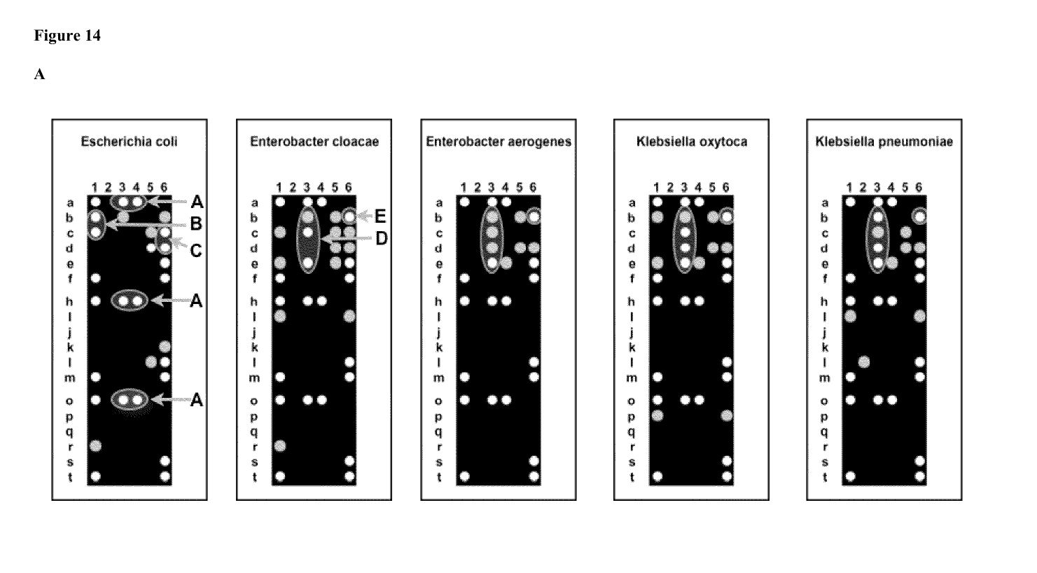

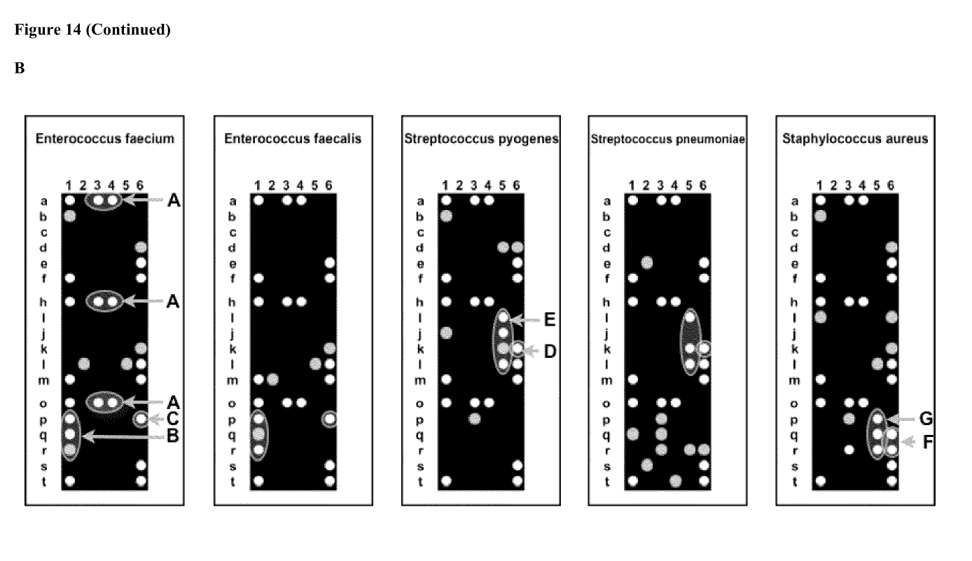

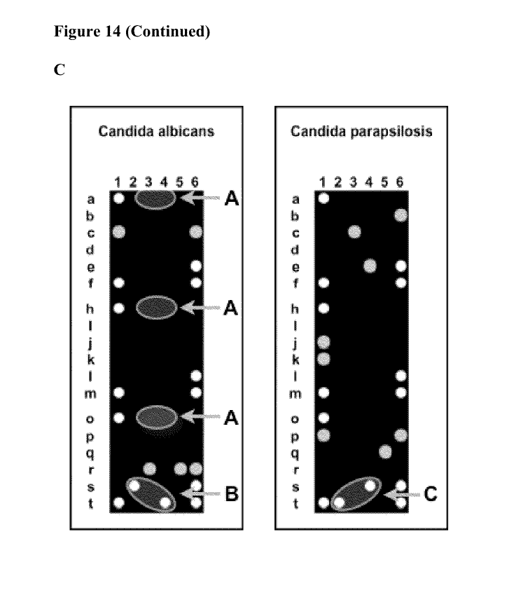

The micro-array is visually evaluated and the results are recorded as no-fluorescence, weak fluorescence, and strong fluorescence and transferred to a graphical form. Representative graphical presentations are depicted in FIG. 14.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments described herein. Such equivalents are intended to be encompassed by the following claims.

What is claimed is:

1. A nucleic acid microarray comprising an array of spots on a solid substrate, wherein the array of spots includes:
a plurality of pathogen-specific spots, each pathogen-specific spot containing a pathogen-specific nucleic acid probe immobilized on the solid substrate;
one or more always-fluorescing spots containing a fluorescent dye immobilized to the solid substrate; and
one or more never-fluorescing spots containing neither a fluorescent dye nor a nucleic acid probe immobilized to the solid substrate;
wherein the one or more always-fluorescing spots and the one or more never-fluorescing spots are positioned such that the array of spots has neither rotational symmetry nor mirror symmetry.

2. The nucleic acid microarray of claim 1, wherein the position of one or more always-fluorescing spots and one or more never-fluorescing spots are such that rotation of the microarray by 90 degrees, 180 degrees or 270 degrees results in at least one always-fluorescing spot being in a position occupied by a never-fluorescing spot in an un-rotated array.

3. The nucleic acid microarray of claim 1, wherein the position of one or more always-fluorescing spots and one or more never-fluorescing spots are such that flipping the microarray on its horizontal or vertical axis results in at least one always-fluorescing spot being in a position occupied by a never-fluorescing spot in an un-rotated array.

4. The nucleic acid microarray of claim 1, wherein the position of one or more always-fluorescing spots and one or more never-fluorescing spots are such that rotation of the microarray by 90 degrees, 180 degrees or 270 degrees results in at least one never-fluorescing spot being in a position occupied by an always-fluorescing spot in an un-rotated array.

5. The nucleic acid microarray of claim 1, wherein the position of one or more always-fluorescing spots and one or more never-fluorescing spots are such that flipping the microarray on its horizontal or vertical axis results in at least one never-fluorescing spot being in a position occupied by an always-fluorescing spot in an un-rotated array.

6. The nucleic acid microarray of claim 1, wherein the position of one or more always-fluorescing spots and one or more never-fluorescing spots are such that rotation of the microarray by 90 degrees, 180 degrees or 270 degrees and flipping the microarray on its horizontal or vertical axis results in at least one never-fluorescing spot being in a position occupied by an always-fluorescing spot in an un-rotated array.

7. The nucleic acid microarray of claim 1, wherein the array of spots further includes one or more positive-control spots containing a nucleic acid probe having a sequence complementary to a positive control nucleic acid.

8. The nucleic acid microarray of claim 7, wherein the positive-control spots contain a nucleic acid probe that is complementary to a conserved region of eubacterial 16S rRNA sequence.

9. The nucleic acid microarray of claim 1, wherein the array of spots is arranged as a rectangular grid of spots.

10. The nucleic acid microarray of claim 9, wherein the rectangular grid of spots contains a plurality of sub-arrays, wherein the distance between adjacent sub-arrays is different than the distance between adjacent spots within the sub-arrays.

11. The nucleic acid microarray of claim 10, wherein the distance between adjacent sub-arrays is greater than the distance between adjacent spots within the sub-arrays.

12. The nucleic acid microarray of claim 11, wherein the distance between adjacent sub-arrays is about 1.5 times the distance between spots within the sub-arrays.

13. The nucleic acid microarray of claim 10, wherein the rectangular grid of spots contains at least four sub-arrays.

14. The nucleic acid microarray of claim 13, wherein the rectangular grid of spots contains nine sub-arrays.

15. The nucleic acid microarray of claim 9, wherein the array of spots is arranged as a square grid of spots.

16. The nucleic acid microarray of claim 9, wherein an always-fluorescing spot is positioned in at least one corner of the rectangular grid of spots.

17. The nucleic acid microarray of claim 16, wherein always-fluorescing spots are positioned in three corners of the rectangular grid of spots and a never-fluorescing spot is positioned in the fourth corner of the rectangular grid of spots.

18. The nucleic acid microarray of any one of claim 1, wherein the plurality of pathogen-specific spots are organized as one or more identification groups, wherein the pathogen-specific nucleic acid probe contained by each spot within an identification group is specific for a target nucleic acid of a related group of pathogens.

19. The nucleic acid microarray of claim 18, wherein the one or more identification groups contain between 4 and 9 spots arranged in a square, rectangle or line.

20. The nucleic acid microarray of claim 18, wherein the related group of pathogens contains less than 30 pathogens.

21. The nucleic acid microarray of claim 20, wherein the related group of pathogens contains less than 8 pathogens.

22. A method of performing a nucleic acid microarray analysis comprising the steps of:
(a) contacting a sample containing fluorescently-labeled nucleic acids with a nucleic acid microarray of any one of claim 1; and
(b) detecting the fluorescence emitted by the spots of the microarray.

23-39. (canceled)

40. A kit comprising a microarray of any one of claim 1.

41-43. (canceled)
